(19) United States
(12) Patent Application Publication
(10) Pub. No.: US 2005/0130311 A1
(43) Pub. Date: Jun. 16, 2005

(54) Detection of impaired fertility

(76) Inventors: John Coley, Rushden Northamptonshire (GB); Fernando Miro, Bedford (GB); Laurence John Aspinall, Bedford (GB); Peter George Murray, Olney Buckinghamshire (GB); Anthony William Dadd, Sharnbrook Bedford (GB); Zhi Gang Zhang, Putnoe (GB); Paul Mundill, Espoo (FI)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/489,908

(22) PCT Filed: Sep. 18, 2002
(86) PCT No.: PCT/GB02/04235

(30) Foreign Application Priority Data
Sep. 18, 2001 (EP) 01307912.4

(51) Int. Cl. G01N 33/00

(52) U.S. Cl. 436/87; 422/61

(57) ABSTRACT

Disclosed is a method of detecting a retarded cycle in a human female subject experiencing same, the method comprising the steps of obtaining a sample of body fluid from the subject on each of a plurality of, but not less than three, days; testing each of the plurality of samples to determine the concentration therein of at least one analyte of significance in the ovulatory cycle; comparing a result determined from said testing with a predetermined threshold value; and, if said determined result is different from the threshold value; declaring the cycle, during which the samples were taken, to be a retarded cycle.

### FIELD OF THE INVENTION

The present invention relates to a method of detecting impaired fertility in a human female subject and/or ascribing or excluding a particular cause to the impaired fertility. The invention also provides a test kit for performing the method.

### BACKGROUND OF THE INVENTION

There are many causes of infertility in women, some “natural” or physiological and some pathological. Eventually, of course, all women reach the end of their reproductive lives and become infertile. This is the menopause, which is defined as the permanent cessation of menstruation due to the loss of ovarian follicular activity (WHO Technical Report Series 670, Geneva 1981: 8-10).

After the menopause a woman is permanently infertile. Beforehand however, there is a “menopause transition” during which a woman progresses from full, normal fecundity to total infertility (i.e. the menopause). The menopause transition is also referred to as the perimenopause. The age at which the menopause transition commences is highly variable, as is the speed of progression. In addition the decline in fertility is unpredictable, in the sense that, even very late in the menopause transition, a woman may occasionally experience fairly normal ovulatory cycles and thus sporadically undergo phases in which she is able to conceive. Thus there is much irregularity in cycle length during the menopause transition. This phenomenon has been well-documented, and it is generally accepted that the onset of the menopause transition is defined by the start of cycle irregularity.

Many changes take place in the concentration and profile of reproductive hormones during the menopause transition. A large number of studies have been reported in the scientific literature. The picture is complicated, but it is true to say that it is accepted that the menopause transition is associated generally with an increase in follicle stimulating hormone (FSH). In order to understand the changes which take place during the menopause transition it is desirable to understand the normal functioning of the ovary.

In healthy, fertile women at the beginning of the menstrual cycle (the follicular phase), the pituitary secretes follicle-stimulating hormone (FSH) which recruits and stimulates the egg-containing follicles in the ovary to grow and produce hormones. The first secretion from the ovary during the cycle is inhibin B, which partially suppresses FSH production. Stimulated by FSH, follicles grow and start to produce significant amounts of estrogens (mainly estradiol) and FSH levels drop further. Eventually, and as a result of the falling FSH concentration, one of the follicles is selected (possibly by virtue of its higher responsiveness to FSH, as well as newly acquired responsiveness to luteinising hormone: LH), its growth is accelerated, and produces a sharp rise in estrogens, which triggers ovulation through the induction of an LH surge. After ovulation (in the luteal phase), the remains of the follicular tissue re-organise into a highly vascularised structure known as the corpus luteum, which secretes large amounts of progesterone, as well as inhibin A and estradiol in lesser amounts. Hormonal levels during this phase peak around day 7 after the LH surge and decrease afterwards until the corpus luteum regresses and a new cycle starts.

However, as a woman gets older ovarian function becomes impaired. Originally, there is an abundant number of follicles recruited, and thus levels of FSH early in the follicular phase are kept relatively low due to the high levels of inhibin B. However, with time the number of follicles recruited decreases, and in consequence FSH levels rise. This decrease in the number of recruited follicles is believed to be the first indication of ovarian ageing.

The direct consequence of the increased FSH levels (also known as the monotropic rise of FSH) is the reduction in the length of the follicular phase of the cycle, due to the faster maturation of the follicles, but menstrual regularity is not disturbed. Reduced fertility has been associated with this elevation of FSH. As FSH levels increase, the follicular phase of the cycle is further reduced. Another fundamental change involved in the process of ovarian ageing is the disruption of menstrual regularity. During this period occur some cycles of relatively long duration, which become more frequent as well as longer.

Fertility (or, more properly, fecundity) is believed to decrease in association with the monotropic rise of FSH, but is even more pronounced after the onset of cycle irregularity. Nevertheless, it is impossible to rule out the existence of fertile cycles once menstrual irregularity has started. Thus impaired ovarian function, as understood above, is due to ovarian ageing which is itself the basis for some of the hormonal changes associated with the menopause transition. For reviews of the subject the reader is referred to the following: Fortune (1994) Biology of Reproduction 50:225-32; Klein et al (1996) Journal of Clinical Endocrinology and Metabolism 81:1038-45; and Prior (1998) Endocrine Reviews 19:397-428.

The menopause transition is associated not only with declining fertility, but with many other symptoms and potential health problems. Thus, there are several reasons why a woman might wish to know whether or not she has commenced the menopause transition. These include: reassurance (a woman may take comfort in knowing that there is a physiological basis for physical symptoms or emotions which are being experienced); to enable an informed decision to be made about whether or not it is appropriate to commence hormone replacement therapy; and whether or not to make ‘lifestyle’ changes, such as commencing an exercise regime or particular diet (e.g. one rich in phytoestrogens) so as to prevent or reduce the likelihood of osteoporosis and related conditions.

There are currently available a number of commercial test kits (e.g. from Genua, N.Y. 14305, USA), use of which is claimed to identify subjects who are post-menopausal (i.e. have gone “through the menopause” and completed the menopause transition). The use of the kit requires the performance of two tests, seven days apart, on urine samples from the subject. If the concentration of FSH exceeds 20 mIU/ml urine in both urine samples, then the subject is declared post-menopausal.

However, as noted above, subjects in the menopause transition may undergo sporadic periods in which their ability to conceive is near normal, and during such periods the absolute and/or relative hormone concentrations may well appear representative of a woman who has not yet commenced the menopause transition. If the prior art kit is used during such periods, it will provide data which, whilst accurate at the time, are extremely misleading in the context of the woman's overall reproductive status.

As a result of the foregoing technical difficulties, it has not so far proved possible to provide a test method which can reliably, and unambiguously, identify whether a particular subject has commenced the menopause transition. Similarly, it has not so far proved possible to provide a test method which can reliably, and unambiguously, ascribe a state of impaired ovarian function as being due to the menopause transition as opposed to any other cause.

All publications mentioned in this specification are specifically incorporated herein by reference.

### SUMMARY OF THE INVENTION

The present inventors have discovered that cycle length irregularity, apparent late in the menopause transition, is due mainly to the occurrence of what may be termed “retarded cycles”. The precise characteristics of a retarded cycle tend to vary, according to the stage of the menopause transition at which the retarded cycle is experienced. A retarded cycle is a cycle with an atypically long follicular phase (relative to normal cycles), with an extended lag phase in follicular development which often (but not inevitably) compromises the quality of the follicle and possibly inhibits ovulation and which, the present inventors believe, is essentially uniquely associated with the menopause transition. In general the inventors have found that, as the menopause transition progresses, the percentage of cycles which are retarded increases, as does the magnitude of the retardation (with a follicular phase of up to 30 days or more in later cycles).

Accordingly, in a first aspect the invention provides a method of detecting a retarded cycle in a human female subject experiencing same, the method comprising the steps of: obtaining a sample of body fluid from the subject on each of a plurality of, but not less than three, days; testing each of the plurality of samples to determine the concentration therein of at least one analyte of significance in the ovulatory cycle; comparing a result determined from said testing with a predetermined threshold value; and, if said determined result is different from the threshold value, declaring the cycle, during which the samples were taken, to be a retarded cycle.

The present inventors believe that such retarded cycles are symptomatic of, and essentially unique to, the menopause transition so that if a woman experiences a retarded cycle it is possible to say with certainty that the woman has commenced the menopause transition: the occurrence of the first retarded cycle is an indication that an irreversible change has taken place, and the subject is now irreversibly embarked upon the menopause transition.

Accordingly in a second aspect the invention provides a method of identifying that a subject who experiences, or has experienced, a retarded cycle, is commencing or has commenced, as appropriate, the menopause transition, the method involving the same steps as defined in the first aspect of the invention.

In addition the invention provides, in a third aspect, a method of identifying impaired ovarian function in a human female subject as being caused by a retarded cycle (i.e. as being due to the menopause transition), the method involving the same steps as defined in the first aspect of the invention.

Those skilled in the art will appreciate that it is not necessary, in order to perform the invention, that the absolute analyte concentration in the samples be determined, and determination of relative concentration (e.g. relative to the selected threshold value) may be adequate. Accordingly, where the specification refers to determination of the “concentration” of an analyte, the expression should be interpreted broadly.

Further, the inventors have found that a retarded cycle is frequently anovulatory. Accordingly, the term “ovulatory” as used herein should not be construed as relating only to those reproductive cycles which include an ovulatory event, but should encompass also those cycles in which there is no ovulatory event, unless the context dictates otherwise.

Moreover, the result determined from the testing may be any value or range of values derivable from the analyte concentrations. For example, in one embodiment, the analyte concentration on each test day is compared with the threshold value and it is noted if the test result for each individual day is significantly different (a “positive” result) or not (a “negative” result) from the threshold value. If a sufficient predetermined number of the individual test results are positive then the cycle may be declared a retarded cycle.

In other embodiments, a mean analyte concentration over a period of several days (consecutive or otherwise) is determined and compared with a reference threshold mean value. In yet other embodiments, a cumulative sum of the analyte concentration may be found and compared with a threshold sum value.

A test result may be positive if the determined result is higher than the threshold value for some analytes, whereas for other analytes the test result may be positive if the determined result is lower than the threshold value.

Thus, a determined result may be “different” to a predetermined threshold value if (a) the threshold value is equal to or lower than a particular value and the determined result exceeds the particular value; or (b) the threshold value is equal to or higher than a particular value and the determined result is below the particular value.

What constitutes a “significant” difference between a determined test value and a predetermined threshold value will depend on the threshold value selected. For example, if the threshold value is in the form of a range, then it may be that any determined test value outside the range is considered significant. Alternatively, if the threshold value is a single value, then it may be that a determined test value will only be considered significantly different if it differs by at least a particular amount, which might be expressed in numerical terms (e.g. at least 5% above or below the threshold value, as appropriate), or may be expressed in statistical terms (e.g. at least 1 standard deviation above or below the threshold).

In general, it is preferred that the days on which the samples are taken are either wholly within the follicular phase of the ovulatory cycle, or wholly within the luteal phase: it is preferred to avoid taking samples in an interval which encompasses a part, or all, of both phases of the ovulatory cycle. Of the two, it is preferred that samples are taken in the follicular phase rather than the luteal phase, since the start of the follicular phase (the first day of bleeding) is easy to determine, is obvious to the subject without the need for any testing, and this consitutes a convenient reference point. The taking of samples during the luteal phase, whilst feasible, is more problematic since the start of the luteal phase (ovulation) may be determined only approximately, and then only readily by measurement of LH concentration (ovulation taking place within 24 hours of the identifiable “LH surge”), which is not such a convenient reference point.

However, some analytes are more readily measured in the follicular phase, whilst others are more readily measured in the luteal phase

The threshold value may be derived from analyses of the concentration of the analyte(s) in question in a large number of subjects, representative of the general population at large (e.g. an arithmetic mean based on data acquired from a population). Such a threshold value may be referred to as a “population-derived” threshold. Alternatively, the threshold value may be derived by analysis of the concentration of the analyte(s) in question in previous cycles or previous portion(s) of the current cycle in the particular subject under investigation, (e.g. an arithmetic mean based on data acquired from a single individual). Such a threshold value may be referred to as any “individual” threshold, as it is valid for one individual but is not necessarily appropriate for a different subject. (It should be noted that tests conducted to establish a threshold value do not constitute part of the “testing” of the invention. For example, where the present specification teaches that testing should commence in the period of days 6-9, this disregards any earlier tests which may have been conducted previously in the same cycle for the purposes of establishing a threshold value). In general, population-derived thresholds are preferred, as data from an individual for previous cycles will not always be available. In particular a population-derived threshold is advantageous when the analyte of significance is follicle stimulating hormone.

The analyte of significance in the ovulatory cycle is typically a hormone, the concentration of which is known to fluctuate during the ovulation cycle. Preferred examples include one or more of the following: follicle stimulating hormone (FSH), luteinising hormone (LH), inhibin (especially inhibin B), progesterone or a metabolite thereof and estrogen or a metabolite thereof. In particular, metabolites of estrogen of use in the methods of the invention include estrone-3-glucuronide (“E3G”), estradiol-3-glucuronide, estradiol-17-glucuronide, estriol-3-glucuronide and estriol-16-glucuronide. For the purposes of brevity, where the context permits, the term “estrogen” is intended also to encompass one or more of the aforementioned metabolites of estrogen. E3G is the most preferred estrogen metabolite for the purposes of the present invention. Similarly, a preferred metabolite of progesterone is pregnanediol-3-glucuronide (“P3G”). FSH is conveniently measured in samples taken within the follicular phase, whereas LH and/or P3G are conveniently measured in samples taken within the luteal phase.

The body fluid sampled preferably comprises urine, but other body fluids which may be sampled in the invention include sweat, saliva, lachrymal fluid, vaginal fluid and the like, all of which are relatively accessible. In principle, internal fluids such as blood and plasma/serum could be sampled but are generally not preferred because they can only be accessed readily by invasive techniques.

It will be apparent that the present invention also encompasses the determination of the concentration of more than one analyte. For example, it may be that the determination of a ratio of the concentration of two analytes provides a more informative assay.

Conveniently the same body fluid samples are used for determination of both or all analyte concentrations. Desirably the one or more further analytes are measured in samples of urine from the subject. The one or more further analytes may comprise any analyte whose concentration determination is of value but will normally be an analyte of significance in the ovulation cycle/control of reproduction in human females. For example, the one or more further analytes may comprise a sex hormone such as progesterone or metabolites thereof or estrogen and metabolites thereof.

In a retarded cycle, there is a delay in that point of the cycle in which E3G starts to become elevated, despite a normally functioning pituitary (as evidenced by FSH levels). That is to say, the ovary is slow to respond to the FSH signal, and therefore follicles are slow to produce estrogens, and FSH levels are thus elevated for longer than normal. In a normal ovulatory cycle, E3G starts to become significantly elevated at about day 7 of the cycle (with day 1 being the first day of bleeding), but in a retarded cycle the E3G rise may be delayed by as little as 2-3 days after this (early on in the menopause transition) to as much as 30 days or more (late in the menopause transition).

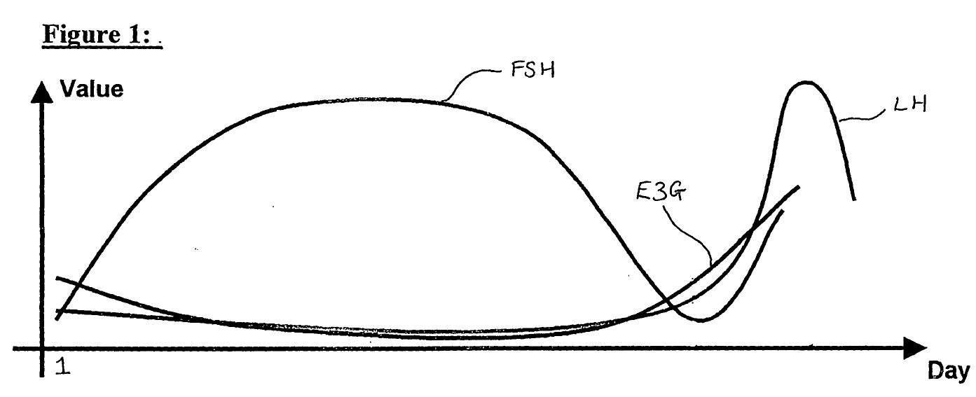

FIG. 1 illustrates typical concentration profiles for FSH, E3G and LH in the follicular phase of a cycle. In a retarded cycle, the “hump” in the FSH profile is extended relative to a normal cycle, and the increase in E3G concentration is delayed.

This delay or retardation can be detected in any one of a number of ways. For example, in the follicular phase of the cycle, the delay can be detected by noting a sustained elevation of FSH above normal levels, or by a correspondingly sustained depression of E3G below normal levels, or by noting a sustained depression of the E3G/FSH ratio, or by any combination of the foregoing.

Alternatively, since the delay has a “knock-on” effect, the retardation of the cycle can also be detected in the luteal phase e.g. by comparing progesterone or derivatives thereof, such as pregnanediol-3-glucuronide (P3G), with the concentration of another analyte, especially E3G.

In one particular embodiment, the invention involves measurement of urinary FSH concentration and determining the mean of the FSH concentration values (f mean) obtained in the period from (and including) day 1 (i.e. first day of bleeding) up to, but excluding, the day of significant E3G rise (i.e. the day of the cycle on which the slope of the plot of urinary E3G concentration against time is maximal). The inventors have devised a number of algorithms, based on retrospective analysis of large amounts of data collected from volunteers, to aid in the detection of retarded cycles. In the above embodiment, a retarded cycle is declared if the FSH mean is greater than a threshold value of 15 m IU/ml and the day of the cycle (with day 1 being the first day of bleeding) on which the maximum urinary FSH concentration occurred (allowing for variation in urine volume) prior to the day of significant E3G rise was later than day 7. This can be presented mathematically by the expression:
If (fmean>15 and fmaxday>7) then output=1
(1 indicating a retarded cycle, 0 indicating a normal cycle), where fmaxday is the day of maximum recorded FSH concentration prior to the day of significant E3G rise.

In another embodiment, the method of the invention involves determination of the fe ratio. The fe ratio is calculated by the sum of all the urinary FSH values divided by the sum of all the urinary E3G values, in the period from (and including) day 1 up to, but excluding, the day of significant E3G rise. If the calculated fe ratio is above a predetermined reference value, then the cycle in question is declared to be a retarded cycle. In the particular assay conditions employed by the inventors, a suitable reference value was found to be 39. The inventors devised an appropriate algorithm, which may be represented by the expression:
If (fe ratio>39) then output=1.

In particular the inventors found that the method of the invention could be improved by combining the two embodiments described above, in a single algorithm:
If (fmean>15 and fmaxday>7) or (fe ratio>39) then output=1.

The threshold level selected, with which an analyte concentration is compared, will depend on the body fluid sample and analyte being tested, the test format and the test reagents used. For example, if immunological techniques are used, the threshold level will depend on the characteristics (e.g. binding affinity) of the antibody molecule employed. The threshold selected will also depend on what are viewed as acceptable false positive (FPR) and false negative (FNR) rates. Generally speaking, both the FPR and FNR should be less than 10%, preferably less than 7%, and ideally no more than 5%, although (as explained in Example 3), an acceptable false negative rate will normally be higher than an acceptance false positive rate. Typically, for samples of urine, the threshold level for FSH will be 10-30 mIU FSH per ml urine, preferably 10-20 m IU FSH/ml, and if the mean FSH level during the follicular phase (or the part thereof in which testing is conducted) exceeds this threshold, the subject is experiencing a retarded cycle.

As stated above, it is a minimum requirement of the invention that tests are conducted on samples taken on at least three different days. Preferably tests are conducted on samples taken on at least four different days, and more preferably tests are conducted on samples taken on at least five different days. Most preferably tests are conducted on samples taken on at least seven days. There is no maximum number of days on which samples may be taken, but there is no particular advantage in testing samples taken on more than 14 days, and conveniently testing is conducted on samples taken on no more than 10 different days.

It is preferred, but by no means essential, that testing is conducted on samples taken on consecutive days. However samples may, for instance, be obtained on alternate days, but this is the minimum desirable sampling frequency. Thus, if a sampling period is considered to extend from the first day on which a sample is obtained to the last day on which a sample is obtained, it is desirable that at least 50% of the days in the sampling period are test days, preferably at least 60%, more preferably at least 70% and most preferably at least 80%.

In one embodiment, where individual days' tests are noted as “positive” or “negative”, the number of positive results (Y) required to make the declaration that a retarded cycle is taking/has taken place depends on the number of days (X) on which testing is conducted. Preferred relationships are set out in Table 1 below (a degree of flexibility exists, in view of the variation in what may or may not be regarded as acceptable false positive or false negative rates):

TABLE 1

| No. of days of | No. of positive results required |
|---|---|
| testing (X) | for Retarded Cycle (Y) |
| 4 | 4 |
| 5 | 4 |
| 6 | 4 |
| 7 | 4 or 5 |
| 8 | 4 or 5 |
| 9 | 6 |
| 10 | 6 or 7 |
| 14 | 7 |

As will be apparent to those skilled in the art, the method of the invention may find particular application amongst women whose ovulatory cycles are irregular. Assuming that such women menstruate, it will however be possible to assign a “first day of bleeding”, which is significant, because the present inventors have found that, for optimum efficiency and/or accuracy, at least in some embodiments, it is necessary for the testing regimen to be conducted within a reasonably defined timescale relative to the first day of bleeding. Thus in a preferred embodiment, especially where the analyte to be measured comprises urinary FSH and/or urinary E3G, assuming day 1 to be the first day of bleeding, it is preferred for the first day of sampling to be conducted in the period of days 5-10, most preferably in the period of days 6-9. More preferably sampling is initiated, and conducted on at least two days, in the period of days 6-9.

For testing of urinary FSH concentrations, when the sampling period is restricted to five days, it is preferred for the sampling to be conducted in the period days 8-13. When the sampling period is seven days, it is preferred for the sampling to be conducted in the period days 7-15 (more preferably days 8-14), and when the sampling period is 10 days, it is preferred for the sampling to be conducted in the period days 6-16. Generally similar sampling periods are suitable for testing of urinary E3G concentrations, although the “window” is a little wider.

Methods of testing the concentration of FSH, E3G, P3G and the like in various samples of human body fluid are well known to those skilled in the art (e.g. Santoro et al, 1996 Journal of Clinical Endocrinology and Metabolism 81, 1495-1501) and form no part of the present invention.

The foregoing comments in respect of FSH relate to determination of FSH concentration in samples obtained during the follicular phase. However, other analytes are more amenable to measurement in the luteal phase. For example, pregnanediol-3-glucuronide (“P3G”) concentration peaks in the luteal phase, and the inventors have found that measurement of a urinary E3G/urinary P3G ratio in the luteal phase, can provide information suitable for performing the method of the invention. As well as the difficulty presented by the lack of a convenient reference point to indicate the start of the luteal phase, P3G levels tend to be highly variable between different individuals. So, whilst for FSH it is relatively straightforward to define a population-derived reference threshold, this is much more problematic for P3G and it is probably necessary instead to use a threshold value calculated from previous measurements in the particular individual in question. In addition, it appears likely that urinary P3G concentration is more variable than, for example, urinary FSH concentration, as the menopause transition progresses.

Whatever the analyte under test, the testing is preferably performed by an immunological technique (i.e. using one or more antibodies or antigen-binding portions thereof as a specific binding reagent). Conveniently the testing is performed using an immunochromatographic technique (e.g. based on those disclosed in WO 94/04925, WO 95/01128 and EP 0656118), typically using a lateral flow assay device, optionally together with a suitably programmed electronic data processing device.

In a further aspect the invention provides a test kit for performing the methods of the invention, the kit comprising a plurality of test devices, typically “dip-stick” type test devices, for determining the concentration of an analyte (such as FSH and/or E3G) in a sample of body fluid from a human female subject, and reading means for reading the test results. The reading means may be simply a visible mark which appears in a test window provided on the test device. Where the embodiment of method of the invention calls for the testing of two or more analytes, it will be convenient if the test devices are capable of measuring the concentration of the two or more analytes, preferably simultaneously but independently. Suitable test devices are disclosed in, for example, EP 0291194 and EP 0560411.

Alternatively the reading means may be incorporated within, or be otherwise functionally associated with, a programmable electronic data processing device in the form of an electronic monitor, which will interpret the results of the tests provided by the test devices and preferably provide an indication, following analysis of a suitable number of test results, as to whether the subject is experiencing or has experienced, as appropriate, a retarded cycle. Thus a further aspect of the invention provides a programmable electronic data processing device for use in the method aspects of the invention defined above, the device being programmed so as to detect, from the results of analyte concentration tests, the occurrence of a retarded cycle; the device optionally further comprising display means to indicate whether or not a retarded cycle had been detected. A suitable electronic monitor, which may readily be modified for use in the present invention by appropriate programming, is disclosed in WO 99/51989. Conveniently the monitor will be programmed to perform one or more of the algorithms disclosed herein.

Preferably the test devices will be cheap and intended to be disposable following a single use. Desirably the test devices will take the form of immunochromatographic dipsticks, to be immersed in a urine sample. Suitable such test devices are well known to those skilled in the art. Conversely the electronic data processing device, if present, will be relatively expensive and reusable.

The person skilled in the art will appreciate that the methods of the invention cannot be used if the subject in question is taking drugs or other substances (e.g. oral contraceptive, HRT) which will affect the normal profiles of hormone analytes which are required to be measured in the method. Also, sometimes the E3G concentration at the start of a cycle is abnormally high, and this should be taken as an indication that meaningful test data will not be available during the follicular phase. Such abnormally high E3G levels at the start of a cycle are primarily due to multiple follicle stimulation in the preceding cycle, which causes a “carry over” of estrogens into the subsequent cycle and a concomitant depression of FSH levels. It should be noted that events of this type can cause an increase in cycle length, but these are not considered as true retarded cycles within the meaning of the term for the purposes of the present invention, since such cycles are not uniquely associated with the menopause transition.

In a further aspect the invention provides a computerised method of detecting a retarded cycle in a human female and/or a method of identifying whether a human female subject has commenced the menopause transition and/or identifying whether impaired ovarian function in a human female subject is due to the menopause transition, said method involving the use of any one, or any combination of, the algorithms disclosed herein (specifically the algorithms disclosed in Examples 3-6). The invention also provides a computer program for use in the computerised method, and a computerised monitor programmed with the program.

The various aspects of the invention will now be further described by way of illustrative example, and with reference to the accompanying drawings in which:

FIG. 1 illustrates the typical profiles of various hormones in the follicular phase of a cycle;

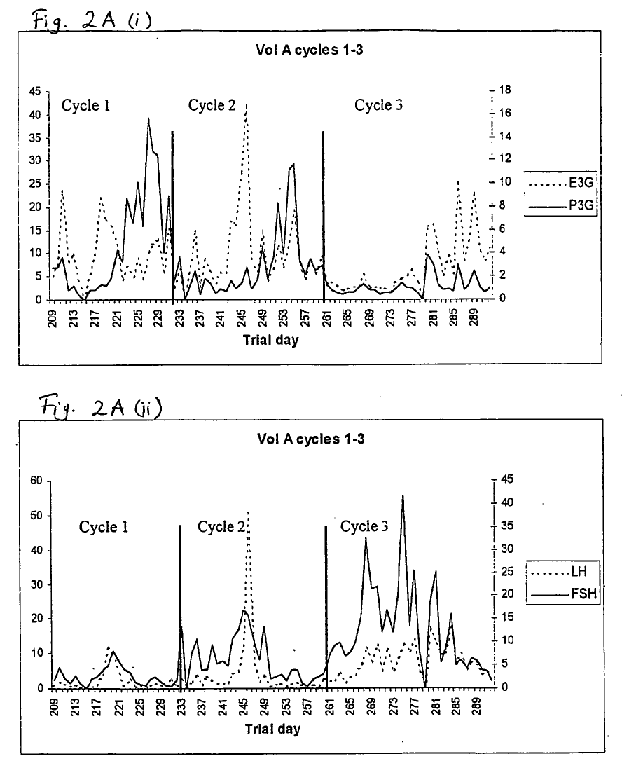

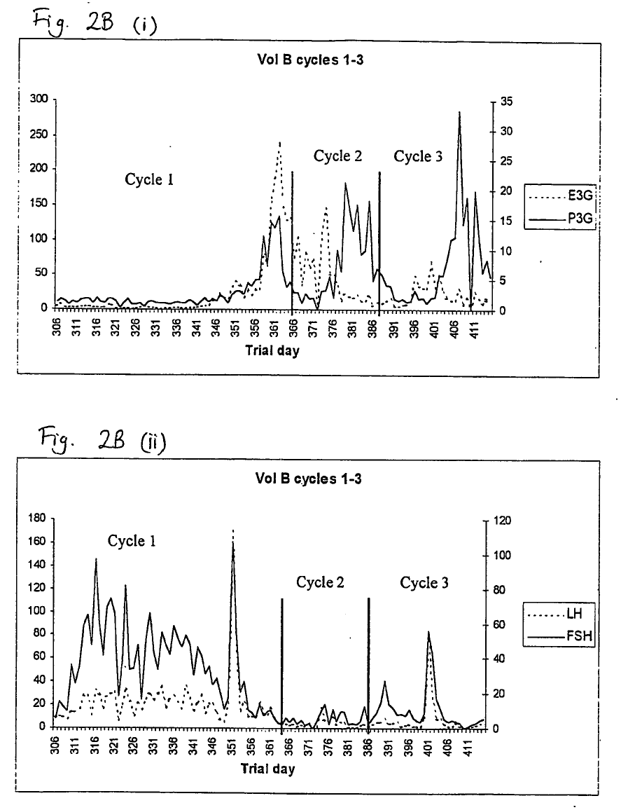

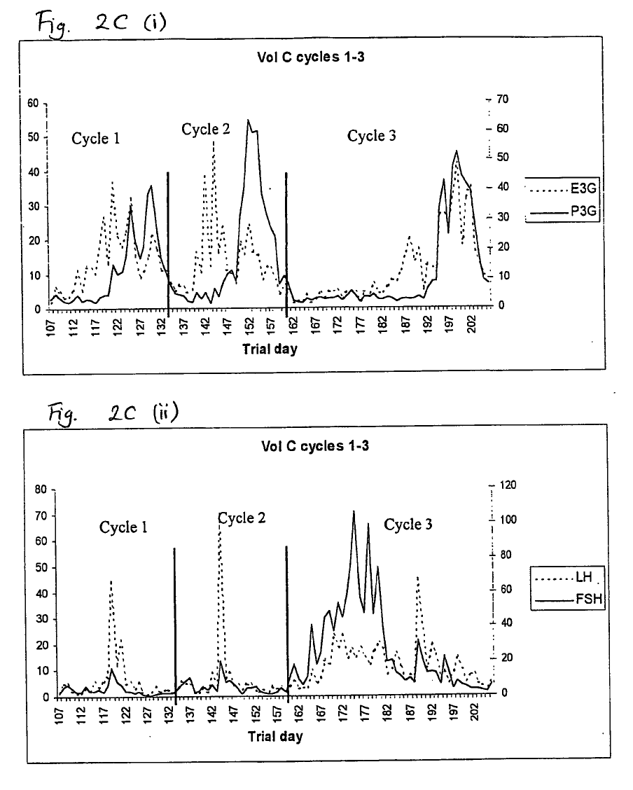

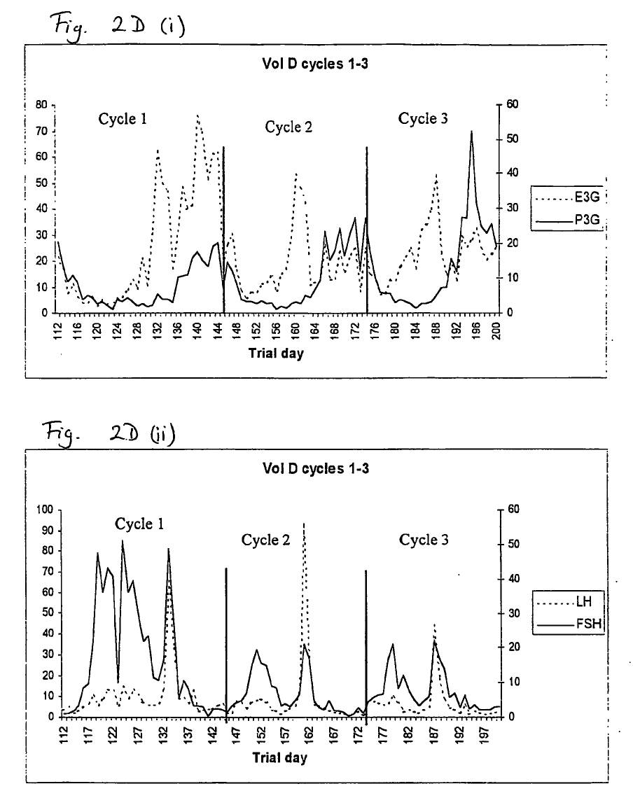

FIGS. 2 A-D illustrate graphs of actual hormone profiles over three cycles in four different subjects; and

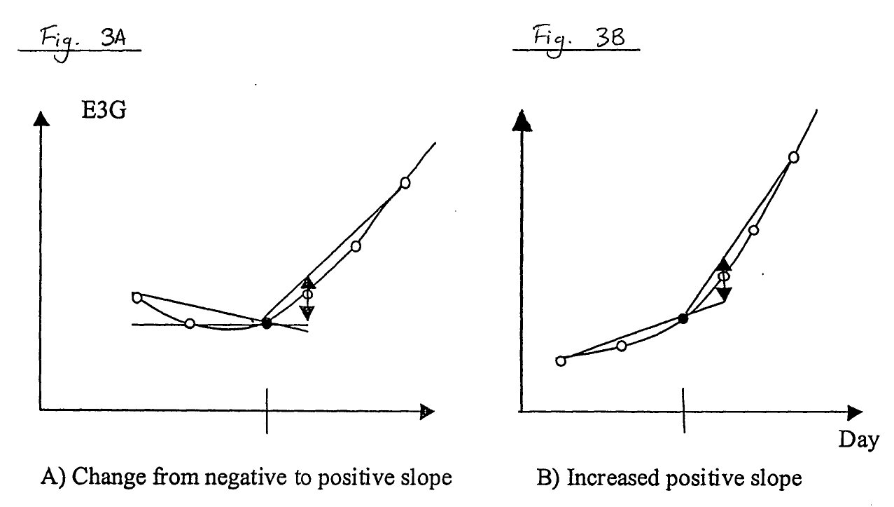

FIGS. 3A & 3B are examples of graphs of E3G concentration against day, illustrating calculation of the day of significant E3G rise.

### EXAMPLES

### Example 1

The inventors gathered data from a confidential study involving a large number of women, aged 30-58, in which daily early morning urine samples were collected over an interval of 6-12 months and stored at 4 to 8° C. (containing sodium azide at 0.1% as preservative) prior to testing.

The samples were analysed to determine the concentration of a number of urinary analytes, including FSH, E3G, P3G and LH. Urinary analyte concentration was determined using an immunoassay technique, run on the AutoDELFIA system which is a high throughput automated system designed to operate up to 24 hours a day, with a minimum of operator intervention. Whilst this facilitated handling of the very large number of samples involved, in principle the same basic assay method could be conducted in a non-automated manner.

The particular assay used to measure FSH concentration involved streptavidin-coated plates, a biotin-labelled anti-FSH monoclonal capture antibody (MAb 4882), and a europium (Eu³⁺)-labelled anti-FSH monoclonal (MAb 5948) to generate the assay signal. These antibodies are not essential to performance of the invention: other anti-FSH monoclonals with similar specificities are commercially available, such as FSH-specific clones 6602 and 6601, available from Medix Biocherica AB, Finland.

The assay protocol was as follows (Wallac Assay Buffer, Wash Buffer Concentrate and Enhancement Solution are reagents specifically developed for DELFIA assays, and are available from Perkin Elner Life Sciences [formerly E.G. & G. Wallac] under the respective products codes 1244-111; 1244-114 and 1244-105):

1. Initially, a solution containing biotin-labelled MAb 4882 (at 1/160 dilution) and Eu³⁺-labelled MAb 5948 (at 1/200 dilution) in Wallac Assay buffer) was prepared and placed in the AutoDELFIA reagent cassette.

2. Streptavidin-coated plates (E.G. & G. Wallac), supplied dry, were loaded into the AutoDELFIA machine and washed with 2×200 μl of wash buffer (wash buffer concentrate obtained from E.G. & G. Wallac).

3. Urine samples under test (25 μl) or standards or controls were dispensed into the wells of the plates.

4. The MAb 4882/MAb 5948 mixture was further diluted 1/100 in assay buffer (from E.G. & G. Wallac) automatically by the AutoDELFIA, giving a final dilution of 1/16,000 for MAb 4882 and 1/20,000 for MAb 5948. 200 μl of the diluted mixture was then added to the wells of the plates.

5. The plate was incubated with shaking for 120 minutes, and then washed with 6×200 μl wash buffer.

6. 200 μl of enhancement solution (E.G. & G. Wallac) was added to each well, the plate shaken for 5 minutes, and the counts read. The concentration values were calculated from a standard curve using the AutoDELFIA Multicalc programme.

Very similar protocols (but employing monoclonal antibodies with appropriate binding specificities) were used to assay urinary LH. Suitable LH-specific antibodies are commercially available from Medix Biochemica, Finland (e.g. monoclonal numbers 5501 and 5503), although the inventors actually used proprietary monoclonals. For the E3G assay, a single antibody competition format was used (described in greater detail elsewhere).

In addition to the urinary analyte data, the ovulatory cycles of the volunteers were also carefully recorded (e.g. cycle length etc.). Retrospective analysis of all these data allowed the inventors to identify women in the volunteer group who had experienced irregular cycles. More detailed analysis revealed that the irregularity was due to the occurrence of cycles with an abnormally extended follicular phase, that is “retarded” cycles in which the usual E3G elevation is delayed, despite a normally functioning pituitary (as evidenced by FSH levels).

By comparison with the other urinary analyte data, it became clear to the inventors that retarded cycles only ever occurred in women who had commenced the menopausal transition, and never occurred in women who had not commenced the transition.

Further the inventors realised that by determining the concentration of one or more urinary analytes on a plurality of days, commencing during the follicular phase of the cycle, it was possible to identify the occurrence of a retarded cycle. The analytes which gave the best discrimination between “retarded” and “normal” cycles were FSH and E3G.

### Example 2

It was evident to the inventors that testing urinary FSH concentration on any single day could not discriminate between “retarded” and “normal” cycles, due to the considerable variability in analyte levels from day to day. On the other hand, it is desirable that the number of days on which tests need to be conducted is as few as possible whilst consistent with providing accurate information. The fewer the number of test days the cheaper the method, and the easier it is for unskilled subjects to perform the testing regime in their homes using simple test kits.

Thus there are a number of variables which the inventors investigated, including: day of cycle on which testing should commence (days 4, 5, 6, 7, 8 or 9); number of test days per cycle (5, 7, 10 or 14 tests/cycle); threshold FSH concentration above which a test would be considered “positive” (10, 15 or 20 mIU/ml); and number of “positive” tests required for a cycle to be declared retarded.

Retrospective analysis of the data collected in the study showed that the best discrimination (in terms of lowest rates of false negatives and false positives) was provided by an assay regime in which:
i) the FSH threshold was set at 15 mIU/ml
ii) the first test day should be day 8 (with day 1 being the first day of bleeding);
iii) there should be 7 tests per cycle; and
iv) at least four positive tests (i.e. returning an FSH concentration above 15 mIU/ml) from the seven tests should be required for a cycle to be declared retarded.

Table 2 below shows representative results for three consecutive cycles (1-3) for four subjects A-D (each of which had commenced the menopause transition), using the testing regime identified above. Testing on seven consecutive days, starting at day 8 of the cycle, using an FSH threshold of 15 mIU/ml, and requiring at least 4/7 test results to be above that threshold for a “positive” result, indicated that, for each subject, one of the three cycles was retarded. Typically, women at this stage of the menopause transition would experience this frequency of retarded cycles, which frequency increases as the women approach the menopause.

For clarity, the profiles of the hormones E3G, P3G, LH and FSH in each of the three cycles are also represented graphically, in FIGS. 2A-D.

FIG. 2A(i) plots the concentration of E3G (broken line) in ng/ml (left hand scale) and P3G (solid line) in g/ml (right hand scale); FIG. 2A(ii) plots the concentration of LH (broken line) in mIU/ml (left hand scale) and FSH (solid line) in mIU/ml (right hand scale); over cycles 1-3 for Subject A.

FIGS. 2B(i) and (ii) show the same data for Subject B, FIGS. 2C(i) and (ii) show the same data for Subject C, and FIGS. 2D(i) and (ii) show the same data for Subject D.

TABLE 2
FSH test - three cycles from each of four peri-menopausal women,
1 cycle is retarded, the other 2 cycles are normal, i.e., ovulatory.

| | | | | | | | | | | No+'s/ | Hormonal |
|---|---|---|---|---|---|---|---|---|---|---|---|
| | | | Test Days | | | | | | | 7 | classification of |
| Person | Cycle | FSH | 8 | 9 | 10 | 11 | 12 | 13 | 14 | tests | the cycle |
| Subject A | 1 | Conc′ (mIU/ml) | 2.1 | 2.4 | 4.0 | 4.9 | 8.1 | 5.7 | 4.1 | | Ovulatory |
| Peri- | | >15 mIU/ml | − | − | − | − | − | − | − | 0+/7 | |
| menopause | 2 | Conc′ (mIU/ml) | 9.3 | 5.3 | 6.0 | 4.9 | 10.7 | 12.5 | 16.8 | | Ovulatory |
| | | >15 mIU/ml | − | − | − | − | − | − | + | 1+/7 | |
| | 3 | Conc′ (mIU/ml) | 15.5 | 32.5 | 21.4 | 21.9 | 12.2 | 16.9 | 12.0 | | Retarded |
| | | >15 mIU/ml | + | + | + | + | − | + | − | 5+/7 | |
| Subject B | 1 | Conc′ (mIU/ml) | 58.5 | 64.7 | 47.7 | 9.7 | 59.7 | 41.5 | 69.2 | | Retarded |
| Peri- | | >15 mIU/ml | + | + | + | − | + | + | + | 6+/7 | |
| menopause | 2 | Conc′ (mIU/ml) | * | 4.52 | 9.8 | 14.3 | 2.4 | 11.0 | 4.1 | | Ovulatory |
| | | >15 mIU/ml | | − | − | − | − | − | − | 0+/7 | |
| | 3 | Conc′ (mIU/ml) | 7.4 | 10.9 | 6.1 | 5.2 | 3.5 | 9.0 | 56.1 | | Ovulatory |
| | | >15 mIU/ml | − | − | − | − | − | − | + | 1+/7 | |
| Subject C | 1 | Conc′ (mIU/ml) | 3.1 | 2.9 | 4.0 | 2.4 | 6.0 | 16.5 | 8.6 | | Ovulatory |
| Peri- | | >15 mIU/ml | − | − | − | − | − | + | − | 1+/7 | |
| menopause | 2 | Conc′ (mIU/ml) | 3.5 | 7.0 | 2.8 | 19.9 | 8.1 | 9.3 | 7.6 | | Ovulatory |
| | | >15 mIU/ml | − | − | − | + | − | − | − | 1+/7 | |
| | 3 | Conc′ (mIU/ml) | 25.0 | 45.0 | 48.8 | 37.1 | 53.6 | 44.8 | 58.6 | | Retarded |
| | | >15 mIU/ml | + | + | + | + | + | + | + | 1+/7 | |
| Subject D | 1 | Conc′ (mIU/ml) | 47.5 | 36.3 | 43.2 | 40.9 | 10.2 | 51.1 | 36.2 | | Retarded |
| Peri- | | >15 mIU/ml | + | + | + | + | − | + | + | 6+/7 | |
| menopause | 2 | Conc′ (mIU/ml) | 15.6 | 14.9 | 9.2 | 8.6 | 3.3 | 4.1 | 3.2 | | Ovulatory |
| | | >15 mIU/ml | + | − | − | − | − | − | − | 1+/7 | |
| | 3 | Conc′ (mIU/ml) | 12.3 | 7.8 | 5.1 | 3.3 | 4.9 | 6.0 | 22.3 | | Ovulatory |
| | | >15 mIU/ml | − | − | − | − | − | − | + | 1+/7 | |

(* samples were not collected - no hormone data)

### Example 3

The collected data showed that, in a retarded cycle,
(a) the average FSH concentration is high throughout most of the follicular phase, and that the FSH peak day is unusually late; and
(b) production of E3G is suppressed during the period of elevated FSH in the follicular phase.

Detailed statistical analysis of the collected data allowed the inventors to draw up two algorithms, defining a retarded cycle, respectively embodying observations (a) and (b) above.

The first algorithm is:
If (fmean>15 and fmaxday>7) then output=1
(1 being a retarded cycle, 0 being a normal cycle).

Fmean is the average (in mIU/ml) of FSH values from day 1 of the cycle up to, but excluding, the day on which there is a significant rise in E3G (or “ERiseDay”, which is the day [prior to occurrence of the LH maximum], on which the slope of the plot of E3G is maximal). (The inventors devised another algorithm which could be used to detect retrospectively the first sustained significant increase in E3G concentration—this is described in Example 5 below). Fmaxday is simply the day of the cycle on which, allowing for urine volume variation, there is a maximum of FSH. (Urine volume variation can be allowed for by several methods; a convenient method is to calculate a ratio of FSH to some other analyte, the concentration of which is also affected by variation in urine volume. A suitable analyte for this purpose is P3G, which is generally present at a reasonably constant, low level during the follicular phase.)

The second algorithm is:
If (fe ratio>39) then output=1

The fe ratio is sum of FSH values divided by sum of E3G values, in the period from day 1 up to, but excluding, the day on which there is a significant rise in E3G.

The inventors found that, by combining the two algorithms as separate clauses within a single algorithm, the test could be made more robust (e.g. the fe ratio threshold value could be set anywhere between 37 and 51, and still provide acceptable rates of false negatives and false positives). It will be apparent that, in the context of the present invention, a false negative result is not unduly worrying—the testing would normally be continued over the course of three or four cycles, so that one false negative may well be offset by a true positive result for a later cycle. Conversely, a false positive result should ideally be avoided, since this could cause unnecessary concern in the individual. Accordingly, the various criteria may well be set so that the acceptable false negative rate is higher (e.g. 10%) than the acceptable false positive rate (e.g. 5%).

### Example 4

This example illustrates how measurements of urinary E3G concentration alone can be used to detect the occurrence of a retarded cycle.

Urine samples were collected from a large group of volunteers as described in Example 1. The samples were analysed retrospectively and a number of analytes assayed, including E3G.

E3G Assay Method

The assay protocol was as follows (Assay Buffer, Wash Buffer Concentrate and Enhancement Solution are reagents specifically developed for DELFIA assays, and are available from Perkin Elmer Life Sciences [formerly EG & G Wallac] under the respective product codes 1244-111; 1244-114 and 1244-105). Rabbit anti-mouse plates (product code AAAND-0003) and europium-labelled estrone-3-glucuronide were obtained from Perkin Elmer Life Sciences.

1. Two solutions were prepared in assay buffer and placed in the AutoDELFIA reagent cassette; anti-E3G antibody (Mab 4155) was pre-diluted 1/40, and Eu³⁺-labelled estrone-3-glucuronide (E3G) was pre-diluted according to the batch of reagent being used.

2. Rabbit anti-mouse plates were loaded into the AutoDELFIA and washed with 2×200 μl wash buffer.

3. Urine samples under test (25 μl) or standards or controls were dispensed into the wells of the plates.

4. The antibody solution was further diluted 1/100 in assay buffer automatically by the AutoDELFIA giving a final antibody dilution of 1/4000. 100 μl of diluted antibody was then added to the wells of the plate.

5. The plate was incubated with shaking for 30 minutes.

6. The E3G-Eu³⁺ conjugate was further diluted 1/100 in assay buffer by the AutoDELFIA. 100 μl of conjugate was then added to the wells of the plate.

7. The plate was incubated with shaking for 30 minutes, and then washed with 6×200 μl wash buffer.

8. 200 μl of enhancement solution (Perkin Elmer Life Sciences) was added to each well, the plate shaken for 5 minutes, and the counts read. The concentration values were calculated from a standard curve using the AutoDELFIA Multicalc programme. By statistical analysis of the data, the inventors were able to prepare an algorithm to detect retarded cycles.

A number of different E3G testing regimes were devised by the inventors, from the results of which tests it was possible to discriminate between retarded cycles and normal cycles in the data collected from the volunteers.

A simple testing regime involves measuring the concentration of urinary E3G on each of days 6-15 of the cycle (i.e. for 10 consecutive days). A reference threshold of 10 ng/ml E3G was adopted. If more than 3 test results in the 10 day testing interval were above this threshold, then the cycle is classified as normal (or ovulatory). Conversely, if the urinary E3G concentration is less than or equal to 10 ng/ml on 8 or more days during the 10 day testing interval then the cycle is classified as retarded.

These test criteria were applied to the same cycles 1-3 of the same subjects A-D as shown in Table 2, and the results are shown below in Table 3. It will be noted that the FSH testing method used in Example 2 and the E3G testing method described above give results in complete agreement as to classification of cycles as being retarded or normal.

Reference is also made to FIGS. 2A-D, mentioned previously, which show the E3G profiles for cycles 1-3 in the four subjects A-D respectively.

TABLE 3
E3G test - three cycles from each of four peri-menopausal women,
1 cycle is retarded, the other 2 cycles are normal, i.e., ovulatory.

| | | | | | | | | | | | | | | Hormonal |
|---|---|---|---|---|---|---|---|---|---|---|---|---|---|---|
| | | | Test days | | | | | | | | | | No+'s/ | classification |
| Person | Cycle | E3G | 6 | 7 | 8 | 9 | 10 | 11 | 12 | 13 | 14 | 15 | 10 | of the cycle |
| Subject A | 1 | Conc (ng/ml) | 3.9 | * | 5.6 | 9.6 | 21.8 | 17.0 | 15.9 | 11.7 | 4.2 | 7.1 | | Ovulatory |
| Peri- | | >10 ng/ml | − | | − | − | + | + | + | + | − | − | 4+/10 | |
| Menopause | 2 | Conc (ng/ml) | 2.9 | 8.9 | 5.8 | 3.4 | 5.7 | 5.8 | 17.5 | 16.0 | 27.5 | 41.9 | | Ovulatory |
| | | >10 ng/ml | − | − | − | − | − | − | + | + | + | + | 4+/10 | |
| | 3 | Conc (ng/ml) | 2.3 | 2.7 | 2.7 | 5.3 | 2.5 | 2.7 | 2.5 | 2.6 | 1.5 | 4.7 | | Retarded |
| | | >10 ng/ml | − | − | − | − | − | − | − | − | − | − | 0+/10 | |
| Subject B | 1 | Conc (ng/ml) | 3.4 | 4.2 | 5.2 | 5.5 | 3.2 | 4.5 | 3.2 | 3.1 | 6.9 | 7.3 | | Retarded |
| Peri- | | >10 ng/ml | − | − | − | − | − | − | − | − | − | − | 0+/10 | |
| Menopause | 2 | Conc (ng/ml) | 58.8 | 71.4 | * | 110 | 146 | 51.9 | 49.1 | 34.2 | 12.0 | 24.7 | | Ovulatory |
| | | >10 ng/ml | + | + | | + | + | + | + | + | + | + | 9+/10 | |
| | 3 | Conc (ng/ml) | 8.0 | 7.1 | 13.0 | 46.7 | 34.9 | 32.9 | 30.9 | 69.5 | 30.0 | 48.6 | | Ovulatory |
| | | >10 ng/ml | − | − | + | + | + | + | + | + | + | + | 8+/10 | |
| Subject C | 1 | Conc (ng/ml) | 5.71 | 11.1 | 5.3 | 12.4 | 11.6 | 10.3 | 19.9 | 26.5 | 12.9 | 36.9 | | Ovulatory |
| Peri- | | >10 ng/ml | − | + | − | + | + | + | + | + | + | + | 8+/10 | |
| Menopause | 2 | Conc (ng/ml) | 5.6 | 16.3 | 10.5 | 38.3 | 12.4 | 48.3 | 16.2 | 23.8 | 11.4 | 10.3 | | Ovulatory |
| | | >10 ng/ml | − | + | + | + | + | + | + | + | + | + | 9+/10 | |
| | 3 | Conc (ng/ml) | 3.6 | 1.6 | 2.5 | 4.4 | 2.7 | 4.5 | 4.9 | 4.9 | 2.5 | 4.5 | | Retarded |
| | | >10 ng/ml | − | − | − | − | − | − | − | − | − | − | 0+/10 | |
| Subject D | 1 | Conc (ng/ml) | 4.0 | 4.2 | 6.1 | 3.0 | 5.1 | 3.6 | 1.5 | 5.5 | 6.3 | 9.1 | | Retarded |
| Peri- | | >10 ng/ml | − | − | − | − | − | − | − | − | − | − | 0+/10 | |
| menopause | 2 | Conc (ng/ml) | 5.8 | 8.1 | 8.1 | 11.5 | 11.7 | 14.1 | 8.5 | 15.3 | 17.3 | 28.9 | | Ovulatory |
| | | >10 ng/ml | − | − | − | + | + | + | − | + | + | + | 6+/10 | |
| | 3 | Conc (ng/ml) | 12.5 | 12.6 | 18.0 | 21.8 | 24.7 | 17.1 | 31.8 | 34.0 | 39.8 | 52.5 | | Ovulatory |
| | | >10 ng/ml | + | + | + | + | + | + | + | + | + | + | 10+/10 | |

(* samples were not collected - no hormone data)

The inventors also explored the outcome when using: different E3G threshold concentrations (6, 7, 8, 9, 10, 11, 12,15, 18 and 20 ng/ml); different testing intervals (days 6-14, 6-15, 7-14, 7-15, 7-16, 8-14, 8-15, 7-16 and 9-15); and different numbers of days (above the threshold) required for a positive result (3, 4, or 5)

The inventors found that the best performance was obtained using a threshold concentration in the range 8-12 ng/ml. Decreasing the threshold to 6ng/ml led to an unacceptably high false negative rate (i.e. too many retarded cycles incorrectly classified as “normal”), whereas increasing the threshold to 15ng/ml or more led to an unacceptably high false positive rate.

The above protocol requires only a measurement of relative E3G concentration (i.e. relative to the selected threshold concentration) and it is therefore possible to perform the necessary testing using simple “dipstick” assay devices or the like, in which the test result for any particular day can be provided by a simple visual cue (e.g. appearance of a line or visible mark on an assay device, as is well-known in the art). However, other testing regimes can be envisaged in which a more quantitative test result is required. Such more quantitative methods may be more readily performed with the assistance of an electronically programmable data processing device, such as an electronic monitor, comprising data processing means programmed to perform the necessary calculations.

At least two different types of such methods may be envisaged; those using a “fixed” threshold, and those using a “floating” threshold.

a) Fixed Threshold

In an example of a “fixed” threshold method, the cycle is identified as being normal or retarded depending on whether the mean E3G concentration in the test interval is above or below a fixed threshold (e.g. 10 ng/ml). A normal cycle has a mean E3G concentration greater than 10 ng/ml, a retarded cycle has a mean E3G concentration equal to or less than 10 ng/ml.

The following test intervals were analysed: days 6-14, 6-15, 7-14, 7-15, 8-14, 8-15, 9-14 and 9-15. Although there is very little variation in performance between the test windows, the best performance was obtained with a test interval of days 8-15.

b) “Floating Threshold”

This embodiment measures a basal E3G value over days 3-6 of each cycle, and then determines whether or not there is a significant rise in the E3G concentration over the test interval (days 7-17), relative to the basal E3G value.

Two criteria were used to define a significant rise (A or B) below:

Significant Rise (A)

The basal value was determined over days 3-6. If the basal concentration was less than 8 ng/ml, then a value of 8 ng/ml is used. If the determined basal level is higher than 8 ng/ml then the higher concentration is used. This compensates for abnormally low concentrations of E3G which may distort the basal determination. The inventors looked at concentrations of 8, 10 and 12 ng/ml as the minimum concentration for the basal value.

The threshold value was defined as being 25% higher than the basal concentration. Thus, for example, if the basal E3G concentration is 8 ng/ml, then the threshold concentration will be 10 ng/ml. However, if the threshold value determined in this manner is greater than 20 ng/ml, then the maximum value of 20 ng/ml is used as the threshold. This compensates for an abnormally high basal E3G concentration. The cycle is “normal” if there are 4 samples in the test interval which exceed the threshold value.

Significant Rise (B)

The alternative method compares the difference between the mean E3G concentration in the basal window, i.e., days 3-6 and the mean E3G concentration for the test interval, e.g., days 7-17. If the mean concentration of E3G in the test interval is less than 12 ng/ml, and the difference between the mean concentration of the test interval the basal window is less than 5 ng/ml, then the cycle is deemed to be “retarded”; i.e., if [meanE3G conc]₇₋₁₇−[meanE3G conc]₃₋₆<5 ng/ml and [mean E3G conc]₇₋₁₇<12 ng/ml, cycle is retarded.

### Example 5

### E3G Rise Algorithm

The algorithm retrospectively detects the first sustained relatively high change in E3G concentration. E3G rise is a parameter that defines the onset of ovarian response to FSH. This parameter is fundamental in determining the effect of elevated concentrations of FSH on follicular development and the presence or absence of a “retarded cycle”. The unit of the parameter is day.

This parameter uses smoothed values of E3G throughout the menstrual cycles, and measures ‘surge size’ (S) for each day (i) in the cycle having a positive slope, but excluding the last ten days of the cycle.

Sᵢ is based on the change in E3G slope, relative to the current E3G level (i.e. on day i):
S₁=(Right₁₃ slope−maximum(Left_slope))/maximum(E3G)
where
Right_slope=the slope of the line joining the current day (i) with the day R days later
Left_slope=the slope of the line joining the current day (i) with the day L days earlier.
Maximum E3G=maximum value of E3G (≧2 ng/mL) (if the E3G concentration does not exceed 4 ng/ml then the value of 4 ng/ml is used).

Illustrative examples are shown in FIGS. 3A & 3B, in which the in-filled circles correspond to the current E3G level, and the double-headed arrow illustrates the value of (Right_slope−maximum(Left_slope). FIGS. 3A and 3B are graphs of (E3G) against time (days). FIG. 3A illustrates the circumstances of a change in the E3G gradient from a negative value to a positive value (i.e. a point of inflection). FIG. 3B illustrates the situation where there is a significant sudden increase in E3G gradient.

In FIG. 3A, Left_slope is negative and therefore is substituted by zero, corresponding to a horizontal line. In these examples: L=2 and R=3; so S depends on the current level of E3G and its values 2 days earlier and 3 days later.

Although L is fixed at 2, R depends on cycle length. For cycles of a length ≦30 days R=3. R increases by 1 for each 5 day increase in cycle length up to 10 for cycle length >60. The latter feature accounts for longer cycles tending to have longer sustained E3G rises.

The divisor, maximum(E3G), gives greater weight to slope changes at low E3G concentrations provided these exceed 4 ng/mL to avoid undue sensitivity.

The E3G rise day is then defined as the first day in the cycle for which: Sᵢ>minimum (0.65*maximum(S), 0.25), i.e., when Sᵢ is either >0.25 or >0.65×its maximum value over the cycle. The latter condition provides adaptation to the cycle, ensuring that a surge is always defined.

### Example 6

This example relates to detection of a retarded cycle by measurement of one or more analytes in the luteal phase of the cycle.

The most suitable markers in the luteal phase for identifying retarded cycles are (preferably) LH and/or (less preferably) E3G/P3G. The timing of the preferred test interval is broadly similar whether measuring LH concentration or the ratio of E3G and P3G concentrations, and the inventors here describe two methods for each marker: 1) counting the number of individual test results over the threshold; and 2) measuring the mean concentrations or ratio during the test interval and comparing it to a threshold value.

The test interval is from the day of LHmax+A to the day of LHmax+B

e.g., A=5, B=16 and the day of LHmax=day 14 of the cycle, then the test interval is days 19-30, i.e., a 12 day test interval.

The inventors tested the following combinations of A and B:

| A | B |
|---|---|
| 3 | 10 |
| 3 | 12 |
| 3 | 14 |
| 3 | 16 |
| 4 | 10 |
| 4 | 12 |
| 4 | 14 |
| 4 | 16 |
| 5 | 10 |
| 5 | 12 |
| 5 | 14 |
| 5 | 16 |

### Example 6A

### E3G/P3G Ratio

The E3G assay method has been described previously. The inventors assayed P3G in urine samples as described below.

P3G assay method

The assay protocol was as follows (Assay Buffer, Wash Buffer Concentrate and Enhancement Solution are reagents specifically developed for DELFIA assays, and are available from Perkin Elmer Life Sciences [formerly EG & Wallac] under the respective product codes 1244-111; 1244-114 and 1244-105). Rabbit anti-mouse plates (product code AAAND-0003) and europium-labelled pregnanediol-3-glucuronide (custom synthesis) were also obtained from Perkin Elmer Life Sciences.

1. Two solutions were prepared in assay buffer and placed in the AutoDELFIA reagent cassette; anti-P3G antibody (Mab 5806) was prediluted 1/40, and Eu³⁺-labelled pregnanediol-3-glucuronide (P3G) was pre-diluted according to the batch of reagent being used.

2. Rabbit anti-mouse plates were loaded into the AutoDELFIA and washed with 2×200 l wash buffer.

3. Urine samples under test (25 l) or standards or controls were dispensed into the wells of the plates.

4. The antibody solution was further diluted 1/100 in assay buffer automatically by the AutoDELFIA giving a final antibody dilution of 1/4000. 100 l of diluted antibody was then added to the wells of the plate.

5. The plate was incubated with shaking for 30 minutes.

6. The P3G-Eu³⁺ conjugate was further diluted 1/100 in assay buffer by the AutoDELFIA. 100 l of conjugate was then added to the wells of the plate.

7. The plate was incubated with shaking for 30 minutes, and then washed with 6×200 l wash buffer.

8. 200 l of enhancement solution (Perkin Elmer Life Sciences) was added to each well, the plate shaken for 5 minutes, and the counts read. The concentration values were calculated from a standard curve using the AutoDELFIA Multicalc programme.

Those skilled in the art will appreciate that there is no necessity to measure P3G (or any other analyte) in this particular way, and methods of analyte measurement per se do not form any part of the invention. For example, known methods for the determination of E3G and P3G include publications by Stancyzk et al., (1980. Amer. J. Obstetrics and Gynecology, 137, 443-450), and Weerasekera et al., (1983. J. Steroid Biochem. 18, 465-470).

The E3G/P3G ratio is simply the arithmetic ratio of the two hormone concentrations, with the units of ng/ml for E3G and μg/ml for P3G.

$$i . e . , \frac{E3G ng \text{/} ml}{P3G µg \text{/} ml}$$

Method 1—Number of Tests Above a Threshold

A cycle is confirmed as retarded if there are n or more days in the test interval with a E3G/P3G ratio greater than T; i.e., E3G (ng/ml)/ P3G (μg/ml)>T on ≧n days in the test window=retarded cycle

The inventors tested threshold ratio values (7) of 2, 3 and 4, and the number of tests (n) required to be greater than the threshold of 2, 3, 4 and 5 tests in the test interval.

The most successful combinations were:
i) A=3, B=16, i.e., 14-day test interval, number of days greater than threshold (n)=2, and threshold ratio (7)=4; and
ii) A=5, B=16, i.e., 12-day test interval, number of days greater than threshold (n)=3, and threshold ratio (1)=3
   Method 2—Mean Value of the Ratio During the Test Interval

A cycle is confirmed as retarded if the mean value of the E3G/P3G ratio during the test interval is greater than threshold ratio (7).

i.e., mean(E3G ng/ml)/P3G (μg/ml) for the days in the test window >T=retarded cycle

The most successful combinations were:
i) A=3, B=16, i.e., 14-day test interval, mean ratio greater than a threshold (7)=3; and
ii) A=5, B=14, i.e., 10-day test interval, mean ratio greater than a threshold (7)=3

This “mean” method was applied to the data obtained during cycles 1-3 of the four subjects A-D used in the previous examples. The results are shown below in table 4.

TABLE 4

| | Mean E3G/P3G ratio in test window | | |
|---|---|---|---|
| | (LHmax day + 3 to + 10) | | |
| | (threshold ratio = 3) | | |
| Subject | Cycle 1 | Cycle 2 | Cycle 3 |
| A | 0.9 | 1.9 | 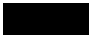 |
| B | 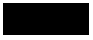 | 1.6 | 1.1 |
| C | 0.9 | 0.6 | 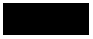 |
| D | 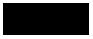 | 1.1 | 1.1 |

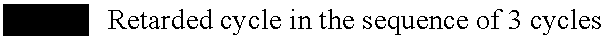

Note
that the rule fails to identify cycle 3 as retarded for subject C

### Example 6B

### LH

Method 1—Number of Tests Above a Threshold

The same range of testing intervals as for the E3G/P3G ratio was explored.

A cycle is confirmed as retarded if there are n or more days with an LH concentration greater than a threshold concentration of L mIU/ml.

i.e., LH concentration >L on ≧n days in the test window=retarded cycle

The inventors tested threshold concentration (L) values of 3, 4, 5 and 6 mIU/ml, and the number of tests (n) required to be greater than the threshold of 2, 3, 4 and 5 tests in the test window.

The most successful combinations were:
i) A=3, B=10, i.e., 8-day test interval, number of days (n) greater than threshold=4, and threshold concentration (L)=4 mIU/ml; and
ii) A=4, B=10, i.e., 7-day test interval, number of days (n) greater than threshold=3, and threshold concentration (L)=4 mIU/ml.
   Method 2—Mean Value of the LH Concentration During the Test Interval

A cycle is confirmed as retarded if the mean concentration of LH during the test interval is greater than a threshold concentration (L).

i.e., mean LH concentration (mIU/ml) for the days in the test window >L=retarded cycle

The most successful combinations were:
i) A=3, B=10, i.e., 8-day test interval, mean LH concentration over the test interval is greater than a threshold concentration (L)=4 mIU/ml; and
ii) A=4, B=10, i.e., 7-day test interval, mean LH concentration over the test interval is greater than a threshold concentration (L)=4 mIU/ml.

This method was applied to the date obtained during cycles 1-3 of the four subjects A-D used in the previous examples. The results are shown below in Table 5.

TABLE 5

| | Mean LH concentration in test window | | |
|---|---|---|---|
| | (LHmax day + 3 to + 10) | | |
| | (threshold concentration = 4 mIU/ml) | | |
| Subject | Cycle 1 | Cycle 2 | Cycle 3 |
| A | 1.1 | 1.5 | 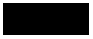 |
| B | 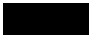 | 3.7 | 3.5 |
| C | 3.4 | 3.4 | 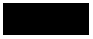 |
| D | 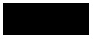 | 2.8 | 3.2 |

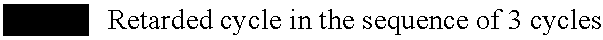

1. A method of detecting a retarded cycle in a human female subject experiencing same, the method comprising the steps of: obtaining a sample of body fluid from the subject on each of a plurality of, but not less than three, days; testing each of the plurality of samples to determine the concentration therein of at least one analyte of significance in the ovulatory cycle; comparing a result determined from said testing with a predetermined threshold value; and, if said determined result is different from the threshold value, declaring the cycle, during which the samples were taken, to be a retarded cycle.

2. A method according to claim 1, wherein the sample of body fluid comprises a urine sample.

3. A method according to claim 1, wherein the analyte of significance comprises one or more of the following: FSH; LH; progesterone or a metabolite thereof; and estrogen or a metabolite thereof.

4. A method according to claim 1, wherein samples of body fluid are taken on consecutive days.

5. A method according to claim 1, wherein each of the plurality of samples are taken on consecutive days.

6. A method according to claim 1, wherein a plurality of samples are taken during the follicular phase.

7. A method according to claim 1, wherein the analyte comprises FSH or E3G and the first of the plurality of samples is taken in the period day 5 to day 10, with day 1 being the first day of bleeding.

8. A method according to claim 7, wherein the first of the plurality of samples is taken in the period day 6 to day 9.

9. A method according to claim 1, wherein each of the plurality of samples is taken within the period of days 6-16, with day 1 being the first day of bleeding.

10. A method according to claim 9, wherein each of the plurality of samples is taken within the period of days 7-15.

11. A method according to claim 9, wherein each of the plurality of samples is taken within the period of days 8-14.

12. A method according to claim 1, wherein a plurality of samples are taken during the luteal phase.

13. A method according to claim 12, wherein the analyte of significance comprises LH, or is a ratio of E3G/P3G.

14. A method according to claim 1, wherein samples are taken on at least 4 days.

15. A method according to claim 14, wherein samples are taken on at least 5 days.

16. A method according to claim 14, wherein samples are taken on at least 7 days.

17. A method according to claim 1, wherein the threshold value is a population-derived threshold.

18. A method according to claim 1, wherein the threshold value is an individual threshold.

19. A method according to claim 1, wherein the analyte of significance comprises FSH and E3G and the cycle is declared to be a retarded cycle if, inter alia, said testing reveals
fmean>X and fmaxday>7, wherein
(i) fmean is defined as the average of FSH concentration values in mIU/ml determined from day 1 of the cycle up to, but excluding, the day on which there is a significant rise in E3G;
(ii) the significant rise in E3G is defined as the day of the cycle (prior to occurrence of the LH maximum) on which the slope of the plot of E3G (against time) is maximal;
(iii) X is a threshold value in the range 10-30 mIU/ml urine;
(iv) and fmaxday is defined as the day of the cycle on which, allowing for urine volume variation, the FSH concentration is maximal.

20. A method according to claim 19, wherein X is in the range 10-20 mIU/ml.

21. A method according to claim 19, wherein X is in the range 12-18 mIU/ml.

22. A method according to claim 1, wherein the analyte of significance comprises FSH and E3G, and the cycle is declared to be a retarded cycle if, inter alia, said testing reveals
fe ratio>Y
wherein fe ratio is the sum of FSH concentration values in mIU/ml divided by the sum of E3G concentration values in ng/ml, in the period from day 1 up to, but excluding, the day of the cycle (prior to occurrence of the LH maximum) on which the slope of the plot of E3G (against time) is maximal, and wherein Y is a number in the range 37-51.

23. A method according to claim 22, wherein Y is in the range 37-41.

24. A method according to claim 22, wherein the cycle is declared to be a retarded cycle if, inter alia, said testing reveals
fmean>X and fmaxday>7, wherein
(i) fmean is defined as the average of FSH concentration values in mIU/ml determined from day 1 of the cycle up to, but excluding, the day on which there is a significant rise in E3G,
(ii) the significant rise in E3G is defined as the day of the cycle (prior to occurrence of the LH maximum) on which the slope of the plot of E3G (against time) is maximal,
(iii) X is a threshold value in the range 10-30 mIU/ml urine,
(iv) and fmaxday is defined as the day of the cycle on which, allowing for urine volume variation, the FSH concentration is maximal.

25. A method according to claim 1, wherein the analyte of significance comprises urinary E3G, and the method involves the determination of a basal E3G value which is the mean urinary E3G concentration of tests conducted in the period of days 3-6 of the cycle.

26. A method according to claim 1, further comprising deeming a menopause transition to have commenced if a retarded cycle is detected.

27. A method according to claim 1, further comprising deeming impaired ovarian function as being due to a menopause transition if a retarded cycle is detected and there are no other apparent causes of impaired function in the subject.

28. A programmable electronic data processing device for use in detecting a retarded cycle in a human female subject experiencing same, the device comprising a computation circuit, responsive to at least three signals, each signal representing the concentration of an analyte of significance to the ovulatory cycle in a sample of body fluid taken on one of three different days, to:
compare at least one of the signals with a predetermined threshold value; and
if said signal is different from the threshold value, declare the cycle during which the samples were taken, to be a retarded cycle.

29. A device according to claim 28, further comprising: receiving means for receiving a test device used for performing an analyte concentration test; and
reading means for reading, from a test device received in the receiving means, the result of an analyte concentration test performed using the test device.

30. A kit for use in detecting a retarded cycle in a human female subject experiencing same, comprising a programmable electronic data processing device according to claim 29, and a plurality of test devices.

31. A device according to claim 28, further comprising display means to indicate whether a retarded cycle has been detected.
